(19) United States
(12) Patent Application Publication
(10) Pub. No.: US 2024/0008725 A1
(43) Pub. Date: Jan. 11, 2024

(54) TUBING AND TUBING ASSEMBLIES FOR ENDOSCOPIC SYSTEM

(71) Applicant: Boston Scientific Scimed, Inc., Maple Grove, MN (US)

(72) Inventor: Khushi Soni, Lexington, MA (US)

(73) Assignee: Boston Scientific Scimed, Inc., Maple Grove, MN (US)

(21) Appl. No.: 18/349,621

(22) Filed: Jul. 10, 2023

Related U.S. Application Data
(60) Provisional application No. 63/388,177, filed on Jul. 11, 2022.

(51) Int. Cl. A61B 1/015; A61B 1/00

(52) U.S. Cl. CPC A61B 1/015; A61B 1/00128; A61B 1/00119

(57) ABSTRACT

Medical devices and methods for using medical devices are disclosed. An example fluid reservoir and tube set arranged and configured to couple to an endoscope for use in an endoscopic procedure includes a fluid supply tube wherein the fluid supply tube includes an extendable region positioned along a length of the fluid supply tube, and a lumen in fluid communication with the fluid reservoir. Further, the extendable region is configured to shift between a first configuration having a first length and a second extended configuration having a second length longer than the first length.

### CROSS-REFERENCE TO RELATED APPLICATIONS

This application claims the benefit of U.S. Prov. Pat. App. No. 63/388,177, filed Jul. 11, 2022, titled TUBING AND TUBING ASSEMBLIES FOR ENDOSCOPIC SYSTEM, which is incorporated herein by reference.

### TECHNICAL FIELD

This disclosure relates generally to medical fluid containers, tubing, and tubing assemblies for fluid delivery, and particularly to extendable tubing utilized with endoscopic systems.

### BACKGROUND

Conventional endoscope devices have been widely used for performing diagnostic and/or therapeutic treatments. Such endoscope devices may be configured to feed fluid to the end of the endoscope for insufflating the inside of the patient at the target site or washing the lens of the endoscope. For example, lens wash and irrigation fluid provide a liquid such as sterilized water at relatively high pressure to spray across and clear debris from the camera lens or target tissue. The water source for lens wash and irrigation typically includes one or more fluid reservoirs with tubing and cap assemblies that establish the plumbing circuit in connection with the endoscope channels and valving to accomplish the desired gas and water functions. Such tubing and tubing assemblies are available in various configurations, which typically involve a water bottle, a cap fitted for the specific bottle, and an array of tubing that is extendable through openings in the cap. The tubing typically is arranged to accommodate a specific configuration of endoscope fittings and valving.

Additionally, certain tubing sets, particularly those that are long and bulky, may be difficult to pack and store. Further, storing and packing tubing sets may become a significant problem when facilities are required store a large quantity of tubing sets. The quantity of tubing sets that a facility may be required to store may increase when the facility has to accommodate for single-use or 24-hour use tubing sets or when a tubing set must be disposed of after coming into contact with a non-sterile surface, such as the floor. Additionally, packaging long and bulky tubing sets is difficult, as the packaging must be large enough to hold the entire tubing set without damaging the product.

This disclosure aims to improve the design of tubing sets, by allowing the tubing sets to be shortened and elongated as desired. Being able to adjust the length of the one or more tubes of the tubing set should allow for tubing sets to be packaged, stored, and handled with more ease, while also making the tubing sets less cumbersome to utilize and setup. There is an ongoing need for improved tubing sets, including tubing sets with adjust length tubing.

### SUMMARY

This disclosure provides design, material, manufacturing method, and use alternatives for medical devices. An example fluid reservoir and tube set arranged and configured to couple to an endoscope for use in an endoscopic procedure includes a fluid supply tube wherein the fluid supply tube includes an extendable region positioned along a length of the fluid supply tube, and a lumen in fluid communication with the fluid reservoir. Further, the extendable region is configured to shift between a first configuration having a first length and a second extended configuration having a second length longer than the first length.

Alternatively or additionally to any of the embodiments above, wherein the extendable region comprises a helical coil in the first configuration.

Alternatively or additionally to any of the embodiments above, wherein the helical coil includes a plurality of coils.

Alternatively or additionally to any of the embodiments above, wherein each individual coil of the plurality of coils is configured to shift between a coiled configuration in the first configuration and a substantially straight configuration in the second extended configuration.

Alternatively or additionally to any of the embodiments above, wherein the fluid supply tube has a circular cross-sectional shape in the extendable region.

Alternatively or additionally to any of the embodiments above, wherein the fluid supply tube is configured to return to the first configuration after being extended to the second extended configuration.

Alternatively or additionally to any of the embodiments above, wherein the helical coil has an outer circumferential shape, and wherein the outer circumferential shape is substantially cylindrical.

Alternatively or additionally to any of the embodiments above, wherein the extendable region comprises a plurality of corrugated folds in the first configuration.

Alternatively or additionally to any of the embodiments above, wherein the plurality of corrugated folds includes a first fold positioned adjacent to a second fold, and wherein a portion of the first fold is nested within a portion of the second fold.

Alternatively or additionally to any of the embodiments above, wherein the extendable region includes a portion of the fluid supply tube which includes an elastic material.

Another example fluid reservoir and tube set arranged and configured to couple to an endoscope for use in an endoscopic procedure includes a fluid reservoir configured to contain a fluid, a gas supply tube, wherein the gas supply tube includes an extendable region positioned along a length of the gas supply tube, and a lumen in fluid communication with the fluid reservoir and a fluid supply tube including a lumen in fluid communication with the fluid reservoir. Further, the extendable region is configured to shift between a first configuration having a first length and a second extended configuration having a second length longer than the first length.

Alternatively or additionally to any of the embodiments above, wherein the extendable region comprises a helical coil in the first configuration.

Alternatively or additionally to any of the embodiments above, wherein the helical coil includes a plurality of coils.

Alternatively or additionally to any of the embodiments above, wherein each individual coil of the plurality of coils is configured to shift between a coiled configuration in the first configuration and a substantially straight configuration in the second extended configuration.

Alternatively or additionally to any of the embodiments above, wherein the extendable region includes a plurality of corrugated folds in the first configuration.

Alternatively or additionally to any of the embodiments above, wherein the extendable region includes a portion of the gas supply tube which includes an elastic material.

Alternatively or additionally to any of the embodiments above, wherein the fluid supply tube includes an additional extendable region positioned along a length of the fluid supply tube.

Alternatively or additionally to any of the embodiments above, wherein at least a portion of the fluid supply tube is co-axial with the gas supply tube and wherein the additional extendable region is co-axial with the extendable region

Another tube set configured to couple to an endoscope for use in an endoscopic procedure includes a fluid reservoir configured to contain a fluid and a tubing assembly coupled to the fluid reservoir. Further, the tubing assembly includes a lens wash tube, a gas supply tube and an irrigation tube. Further, at least one of the lens wash tube, the gas supply tube and the irrigation tube includes an extendable region positioned along a length thereof, and a lumen in fluid communication with the fluid reservoir. Further, the extendable region is configured to shift between a first configuration having a first length and a second extended configuration having a second length longer than the first length.

Alternatively or additionally to any of the embodiments above, wherein the extendable region comprises a helical coil in the first configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 depicts components of an endoscope;

FIG. 2 depicts components of an endoscopic system;

FIG. 3A depicts an endoscopic system wherein the system is activated to deliver air to atmosphere;

FIG. 3B depicts the endoscopic system of FIG. 3A, wherein the system is activated to deliver air to a patient through the patient end of the endoscope;

FIG. 3C depicts the endoscopic system of FIG. 3A, wherein the system is activated to deliver lens wash fluid through the patient end of the endoscope;

FIG. 3D depicts the endoscopic system of FIG. 3A, wherein the system is activated to deliver irrigation fluid through the patient end of the endoscope;

FIG. 4 depicts a portion of an endoscopic system including a container and a plurality of tubes coupled thereto;

FIG. 5 depicts a cross-sectional view of a tube of FIG. 4 taken along line 5-5;

FIG. 6 depicts the endoscopic system of FIG. 4 including one of the plurality of tubes in an extended configuration;

FIG. 7 depicts a portion of an endoscopic system including a container and a plurality of tubes coupled thereto;

FIG. 8 depicts the endoscopic system of FIG. 7 including one of the plurality of tubes in an extended configuration;

FIG. 9 depicts a portion of an endoscopic system including a container and a plurality of tubes coupled thereto;

FIG. 10 depicts the endoscopic system of FIG. 9 including one of the plurality of tubes in an extended configuration;

FIG. 11 depicts a portion of another the endoscopic system including a plurality of tubes in a co-axial configuration.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Although embodiments of the present disclosure are described with specific reference to a bottle (e.g., container, reservoir, or the like) and tube assembly or set, it should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes description of a bottle and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

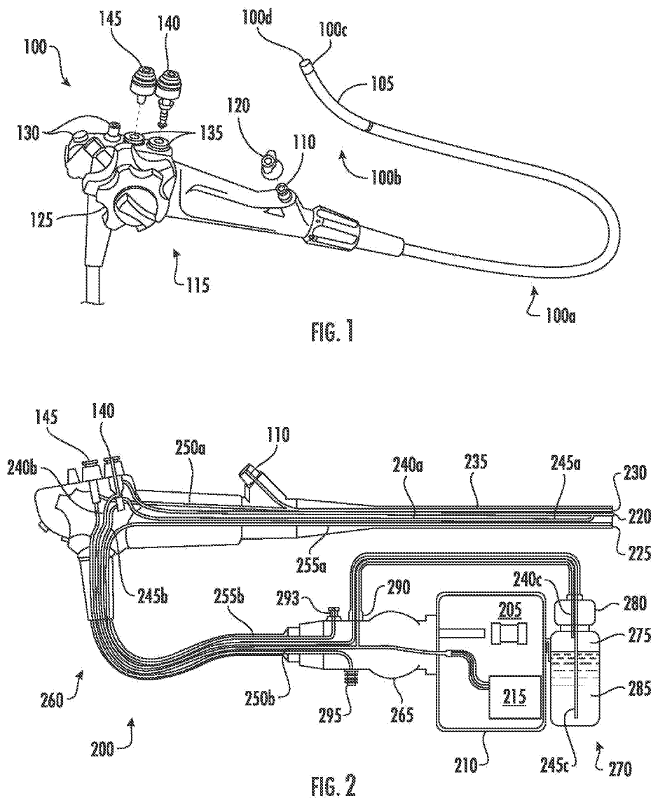

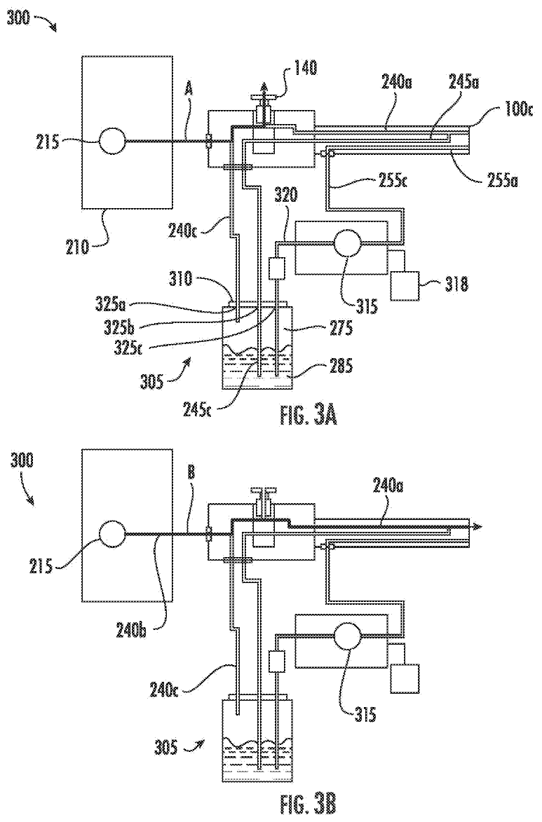

With reference to FIGS. 1-2, an exemplary endoscope 100 and endoscopic system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air or gas feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, also may be included on the end face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A fluid container or reservoir 270 (e.g., water bottle) is fluidly connected to the endoscope 100 through the connector portion 265 and the umbilical 260. A length of gas supply tubing 240c passes from one end positioned in an air gap 275 between the cap 280 (e.g., bottle cap) of the reservoir 270 and the remaining water 285 in the reservoir 270 to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the cap 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. As is discussed in greater detail below, outflow check valves, one-way valves, or backflow prevention mechanisms may be placed in the path of the lens wash supply tubing (or other tubing of system 200, 300) to help prevent water from back-flowing into the reservoir 270 after the water has passed the valve.

A relatively higher flow rate of irrigation water compared to lens wash is typically required, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the cap of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the cap 280 of the water reservoir 270. The vent allows atmospheric air into the water source preventing negative pressure build-up in the water source, which could establish a vacuum that suctions undesired matter from the patient back through the endoscope toward the water source. In some embodiments, outflow check valves, one-way valves, or backflow prevention mechanisms, similar to the lens wash tubing 245c, may be placed in the path of the irrigation supply tubing to help prevent back-flow into the reservoir after water has passed the valve.

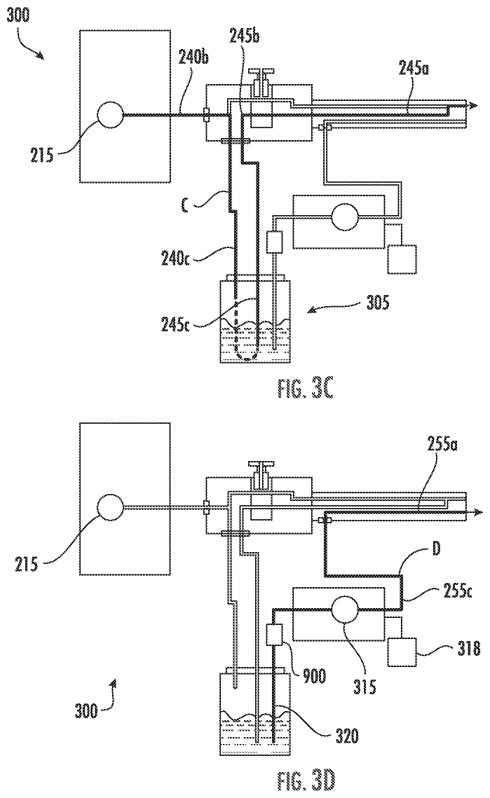

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash and irrigation.

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 245c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 245c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 245c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c without passing through the gas/water valve 140. The irrigation pump pressure may be calibrated, along with the endoscope irrigation feed and supply lines and external tubing, to deliver a certain range of flow rate of the irrigation fluid.

In some embodiments, a variety of tubing configurations may be incorporated into the embodiments disclosed herein, including the tubing of the systems 200, 300. For example, the gas supply tubing 240c, lens wash supply tubing 245c, upstream irrigation supply tubing 320, downstream irrigation supply tubing 255c, or any other tubing in the systems 200, 300 may include one or more features which permit the tubing to elongate and shorten. Designing the tubing components of the systems 200, 300 to elongate and shorten may permit the endoscopic tubing sets to be packaged, stored and manipulated with improved efficiency and ease. For example, designing various tubing components of the systems 200, 300 to elongate and shorten as desired may allow a user to more easily manipulate an endoscope relative to a patient during an endoscopic procedure (e.g., as the user manipulates the endoscope, the tubing components lengthen and shorten as required). Further, designing multiple tubing components of the systems 200, 300 (e.g., a tubing set of the systems 200, 300) to elongate and shorten may permit the tubing set (including gas supply tubing 240c, lens wash supply tubing 245c, upstream irrigation supply tubing 320, downstream irrigation supply tubing 255c, and other tubing), to be packaged in a compact and efficient configuration, thereby saving storage space.

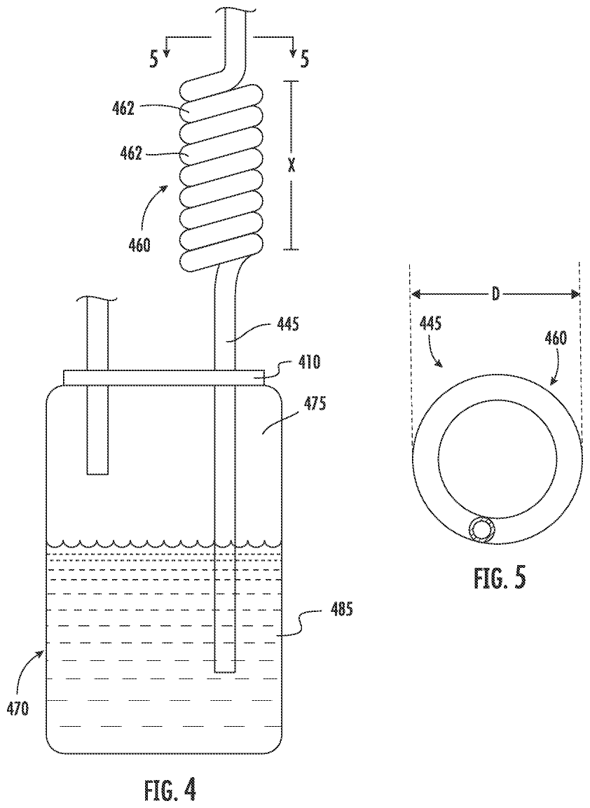

FIG. 4 illustrates an example water reservoir 470 (e.g., water container). The water reservoir 470 may be similar in form and function to the water reservoirs 270, 305 of the endoscopic systems 200, 300, described herein. For example, FIG. 4 illustrates that the water reservoir 470 may include an inner chamber designed to hold varying volumes of water 485. Additionally, FIG. 4 illustrates a cap 410 may be securely fastened to the water reservoir 470. Securement of the cap 410 to the water reservoir 470 may establish an air gap 475 between the cap 410 of the reservoir 470 and the water 485 in the reservoir 470.

FIG. 4 further illustrates a length of gas supply tubing 440 passing from one end positioned in an air gap 475 and through the cap 410 of the reservoir 470. It can be appreciated from FIG. 4 that the gas supply tubing 440 may be similar in form and function to the gas supply tubing 240c described herein. Additionally, FIG. 4 illustrates a length of lens wash tubing 445 having one end positioned within the water 485 of the reservoir 470 and passing through the cap 410 of the reservoir 470. It can be appreciated from FIG. 4 that the lens wash tubing 445 may be similar in form and function to the lens wash tubing 245c described herein. It can be appreciated that the systems 200, 300 may further a length of irrigation tubing (not shown, but similar in form and function to the irrigation supply tubing 320 discussed herein) having one end positioned within the water 485 of the reservoir 470 and passing through an opening in the cap 410 of the reservoir 470. In some embodiments, the irrigation supply tubing and lens wash tubing 445 may source water from the same reservoir 470, however, in other embodiments the irrigation water may be supplied via a pump (e.g., peristaltic pump) from a water source independent (not shown) from the water reservoir 470.

FIG. 4 further illustrates that the lens wash tubing 445 may include an extendable region 460. The extendable region 460 of FIG. 4 may further define a region of the lens wash tubing 445 that is helically coiled. The helically coiled portion of the lens wash tubing 445 may include a plurality of individual, pre-formed coils 462. In an unstressed (e.g., unelongated, unstretched, neutral) configuration such as that illustrated in FIG. 4, the individual coils 462 may be positioned directly adjacent one another. It can be appreciated that the helically coiled configuration illustrated in FIG. 4 may be formed by taking a substantially straight length of lens wash tubing and winding a portion of it into the compact helical coil configuration shown in FIG. 4. The helical coil shown in FIG. 4 has a length X when not extended.

After the lens wash tubing 445 is wound into the helical coil configuration illustrated in FIG. 4, the lens wash tubing 445 may be imparted with a pre-shaped memory. In other words, the lens wash tube 445 may be pre-formed into the helical coil configuration with the coils 462 assuming the coiled, compact configuration whenever the lens wash tube 445 is in a neutral, unstressed condition. Accordingly, the stretching of the lens wash tube 445 (e.g., the lengthening of the extendable region 460) typically does not permanently deform the helical coil shape of the extendable region 460. Rather, the lens wash tube 445 may have an elastic shape memory which may return the tube 445 to its original helically coiled shape when there is no stress on the lens wash tube 445 (e.g., when the lens wash tube 445 is not being extended). However, the lens wash tube 445 may be easily stretched, as when a user pulls an endoscope 100 away from the water reservoir 470.

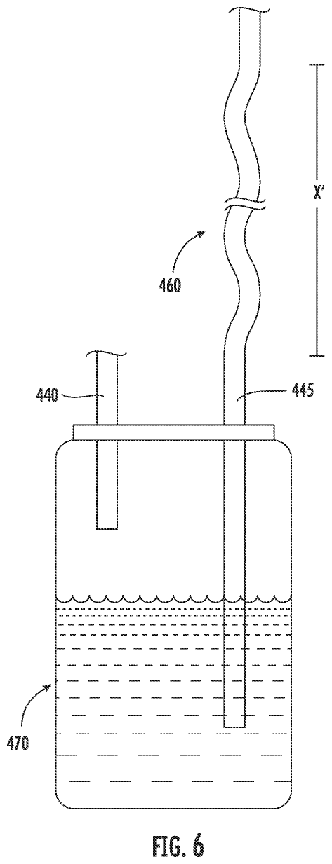

Additionally, the region of lens wash tubing 445 defining the extendable region 460 (and, therefore, each of the individual coils 462) may include a cross-sectional shape. The cross-sectional shape of the region of lens wash tubing 445 defining the helical coils 462 shown in FIG. 4 may be substantially circular (as shown in FIG. 5, discussed below). However, in other examples, the cross-sectional shape of the region of lens wash tubing 445 defining the helical coils 462 may include elliptical, oval, triangular, square, rectangular, other polygonal shapes or combinations thereof.

FIG. 5 illustrates a cross-sectional view of the extendable region 460 taken along line 5-5 of FIG. 4. As shown in FIG. 5, the circumferential shape of the lens wash tubing 445 defining the helical coils 462 may be a circle having an outer diameter D. In some examples, the diameter D may be about 0.10 inches to 1.50 inches, or about 0.15 inches to 1.25 inches, or about 0.20 inches to 1.10 inches, or about 0.25 inches to 1.0 inches, or about 0.50 inches to 0.75 inches. Additionally, in some examples, each of the individual coils 462 defining the helical coil may have approximately the same diameter D along the length X of the extendable region 460. However, in other examples, some individual coils 462 may be larger (or smaller) in diameter than other individual coils 462. Further, while FIG. 4 illustrates the individual coils 462 positioned directly adjacent to one another, in some examples, the coils may be evenly spaced away from one another. Yet in other examples, the individual coils 462 may be unevenly spaced away from one another.

FIG. 6 illustrates the water reservoir 470, gas supply tubing 440 and lens wash tubing 445 of FIG. 4, whereby the extendable region 460 of the lens wash tubing 445 has been lengthened from length X shown in FIG. 4 to a length X′. FIG. 6 represents a configuration in which the helical coils 462 of the extendable region 460 have been stretched (by a user manipulating the endoscope 100, for example, by moving the endoscope 100 further from the water reservoir 405). In some examples, the configuration illustrated in FIG. 6 may be referred to as an elongated or stressed tubing configuration. As discussed herein, it can be appreciated that the extendable region 460 shown in FIG. 6 may return to its helical shape shown in FIG. 4 as a user moves the endoscope 100 closer to the water reservoir 405, thereby permitting the extendable region 460 to shift from a stressed, elongated configuration to an unstressed, neutral (e.g., relaxed) configuration.

It can be appreciated from the above discussion relative to FIGS. 4-6 that regardless of the diameter of the lens wash tubing 445 or the particular material used to construct the lens wash tubing 445, the lens wash tubing 445 could be coiled tightly against itself. Even when tightly coiled, the lens wash tube 445 may still allow for water flow while also allowing the lens wash tubing 445 to be packaged and stored in a tight, compact configuration. Additionally, even when tightly coiled, the lens wash tubing 445 may remain flexible during procedures. With the ability to elongate and shorten, the physician may relocate equipment within the procedure room without fear of the lens wash tubing 445 not being able to reach and/or accommodate a variety of different configurations. Additionally, for more complicated tubing sets, including tubing sets having multiple tubes, helically coiled tubing may improve the effective organization of each individual tube in the tubing set, thus eliminating time wasted untangling the tubes from themselves. The advantages listed herein also apply to tubing components other than the lens wash tubing 445. For example, the advantages listed here apply to the gas supply tubing, lens wash supply tubing, upstream irrigation supply tubing, downstream irrigation supply tubing, multiple tubing sets including more than one type of tubing, or any other tubing or component of systems 200, 300.

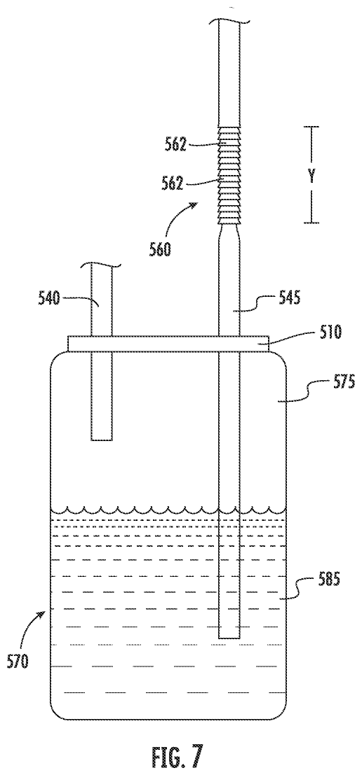

FIG. 7 illustrates an example water reservoir 570 (e.g., water container). The water reservoir 570 may be similar in form and function to the water reservoirs 270, 305 of the endoscopic systems 200, 300, described herein. For example, FIG. 7 illustrates that the water reservoir 570 may include an inner chamber designed to hold varying volumes of water 585. Additionally, FIG. 7 illustrates a cap 510 that may be securely fastened to the water reservoir 570. Securement of the cap 510 to the water reservoir 570 may establish an air gap 575 between the cap 510 of the reservoir 570 and the water 585 in the reservoir 570.

FIG. 7 further illustrates a length of gas supply tubing 540 passing from one end positioned in an air gap 575 and through the cap 510 of the reservoir 570. It can be appreciated from FIG. 7 that the gas supply tubing 540 may be similar in form and function to the gas supply tubing 240c described herein. Additionally, FIG. 7 illustrates a length of lens wash tubing 545 having one end positioned within the water 585 of the reservoir 570 and passing through the cap 510 of the reservoir 570. It can be appreciated from FIG. 7 that the lens wash tubing 545 may be similar in form and function to the lens wash tubing 245c described herein. It can be appreciated that the systems 200, 300 may further a length of irrigation tubing (not shown, but similar in form and function to the irrigation supply tubing 320 discussed herein) having one end positioned within the water 585 of the reservoir 570 and passing through the cap 510 of the reservoir 570. It can be appreciated from that the irrigation tubing may be similar in form and function to the irrigation tubing 320 described herein. While FIG. 7 illustrates that the irrigation supply tubing and lens wash tubing 545 may source water from the same reservoir 570, in some embodiments the irrigation water may be supplied via a pump (e.g., peristaltic pump) from a water source independent (not shown) from the water reservoir 570.

FIG. 7 further illustrates that the lens wash tubing 545 may include an extendable region 560. The extendable region 560 of FIG. 7 may further define a region of the lens wash tubing 545 that includes a plurality of individual, corrugated folds 562 positioned along a length Y of the lens wash tubing 545. In a pre-elongated (e.g., unstressed, neutral) configuration (such as that illustrated in FIG. 7), the individual folds 562 may be positioned directly adjacent to one another, whereby the individual folds 562 may stack on top of each other along the length Y. It can be appreciated that the plurality of corrugated folds 562 illustrated in FIG. 7 may be formed by taking a substantially straight length of tubing and forming the corrugated folds 562 such that each individual fold 562 nests into an adjacent fold 562, thereby forming the compact corrugated (e.g., accordion) folded configuration as illustrated in FIG. 7.

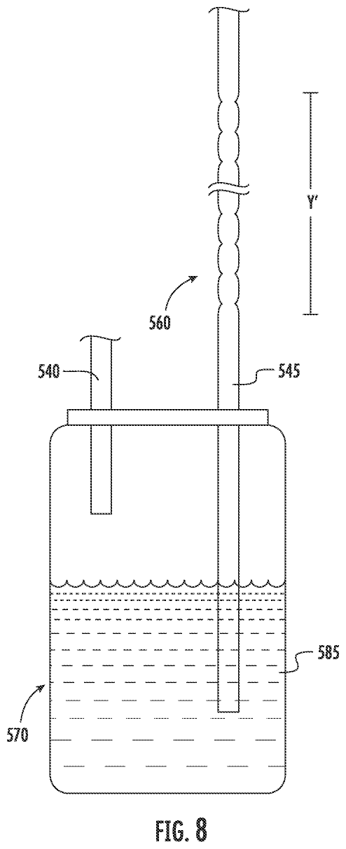

FIG. 8 illustrates the water reservoir 570, gas supply tubing 540 and lens wash tubing 545 of FIG. 7, whereby the extendable region 560 of the lens wash tubing 545 has been lengthened from length Y shown in FIG. 7 to the length Y′. FIG. 8 represents a configuration in which the compact, corrugated folds 562 of the extendable region 560 have been elongated (by a user manipulating the endoscope 100, for example). In some examples, the configuration illustrated in FIG. 8 may be referred to as an elongated or stressed tubing configuration.

In some examples, stretching the extendable region 560 of the lens wash tube 545 may result in a generally permanent lengthening of the extendable region 560 of the lens wash tube 545. In other words, in some examples, a user pulling on the lens wash tube 545 may unfold one or more of the plurality of folds 562, thereby lengthening the extendable region 560. In this example, the folds 562 may be designed to remain unfolded, thereby preserving the lens wash tube 545 in its lengthened configuration, unless the user physically re-compacts the folds back to a compact corrugated folded configuration.

However, in other examples, it can be appreciated that the extendable region 560 shown in FIG. 8 may return to its corrugated configuration shown in FIG. 7 as a user moves the endoscope 100 closer to the water reservoir 570. In other words, in some examples, the individual folds 562 of the extendable region 560 may be imparted with a shape memory which permits them to shift from an elongated configuration (shown in FIG. 8) to back to a folded configuration (shown in FIG. 7). In some examples, the shape memory capabilities of the individual folds 562 of the extendable region 560 may correspond to the shape memory characteristics of the material of the lens wash tube 545.

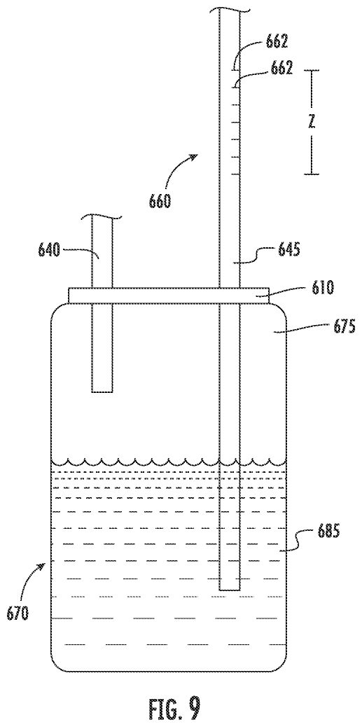

FIG. 9 illustrates an example water reservoir 670 (e.g., water container). The water reservoir 670 may be similar in form and function to the water reservoirs 270, 305 of the endoscopic systems 200, 300, described herein. For example, FIG. 9 illustrates that the water reservoir 670 may include an inner chamber designed to hold varying volumes of water 685. Additionally, FIG. 9 illustrates a cap 610 may be securely fastened to the water reservoir 670. Securement of the cap 610 to the water reservoir 670 may establish an air gap 675 between the cap 610 of the reservoir 670 and the water 685 in the reservoir 670.

FIG. 9 further illustrates a length of gas supply tubing 640 passing from one end positioned in an air gap 675 and through the cap 610 of the reservoir 670. It can be appreciated from FIG. 9 that the gas supply tubing 640 may be similar in form and function to the gas supply tubing 240c described herein. Additionally, FIG. 9 illustrates a length of lens wash tubing 645 having one end positioned within the water 685 of the reservoir 670 and passing through the cap 610 of the reservoir 670. It can be appreciated from FIG. 9 that the lens wash tubing 645 may be similar in form and function to the lens wash tubing 245c described herein. It can be appreciated that the systems 200, 300 may further a length of irrigation tubing (not shown, but similar in form and function to the irrigation supply tubing 320 discussed herein) having one end positioned within the water 685 of the reservoir 670 and passing through the cap 610 of the reservoir 670. It can be appreciated from that the irrigation tubing may be similar in form and function to the irrigation tubing 320 described herein. While FIG. 9 illustrates that the irrigation supply tubing and lens wash tubing 645 may source water from the same reservoir 670, in some embodiments the irrigation water may be supplied via a pump (e.g., peristaltic pump) from a water source independent (not shown) from the water reservoir 670.

FIG. 9 further illustrates that the lens wash tubing 645 may include an extendable region 660. The extendable region 660 of FIG. 9 may further define a region of the lens wash tubing 645 that includes an elastic material positioned along a length Z of the lens wash tubing 645. In a pre-elongated (e.g., unstressed, neutral) configuration (such as that illustrated in FIG. 9), the elastic material of the extendable region 660 may exhibit a flexible, compact, unelongated configuration.

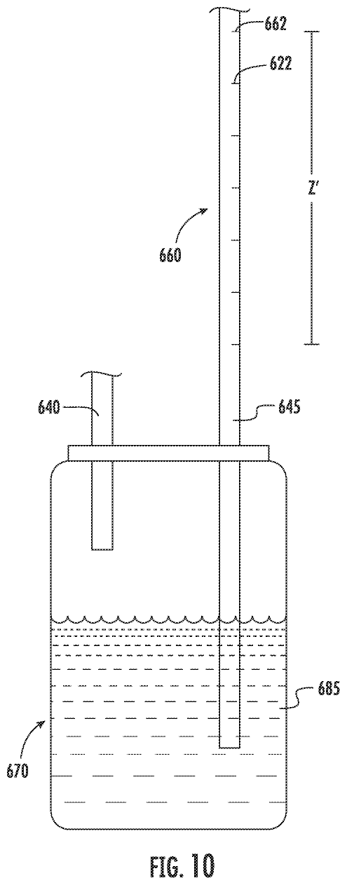

FIG. 10 illustrates the water reservoir 670, gas supply tubing 640 and lens wash tubing 645 of FIG. 9, whereby the extendable region 660 of the lens wash tubing 645 has been lengthened from length Z shown in FIG. 9 to the length Z′. FIG. 10 represents a configuration in which the elastic material of the extendable region 660 has been elongated (by a user manipulating the endoscope 100, for example). In some examples, the configuration illustrated in FIG. 10 may be referred to as an elongated or stressed tubing configuration.

In some examples, stretching the extendable region 660 of the lens wash tube 645 may result in a generally permanent lengthening (permanent deformation) of the lens wash tube 645. In other words, in some examples, a user pulling on the lens wash tube 645 may stretch the elastic material, thereby lengthening the extendable region 660. In this example, the elastic material may be designed to remain permanently lengthened, thereby preserving the lens wash tube 645 in its lengthened configuration. However, in other examples, it can be appreciated that the elastic material used to construct the extendable region of the lens wash tube 645 may have a shape memory and undergo temporary deformation such that it returns to its configuration in FIG. 9 as a user moves the endoscope 100 closer to the water reservoir 670.

It can be further appreciated that the various configurations of the extendable regions 460, 560, 660 of the lens wash tubing 445, 545, 645 described herein may be applied to any of the tubes of system 200, 300. For example, helical coils, corrugated folds, elastic materials, and other extendable features may be incorporated in one or more of the gas supply tubing, lens wash supply tubing, upstream irrigation supply tubing, downstream irrigation supply tubing or any other tubing or component of systems 200, 300.

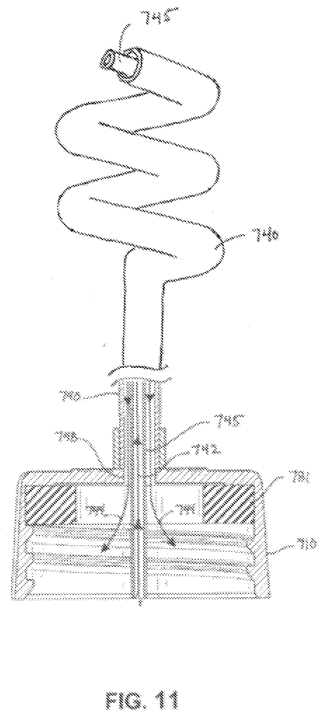

FIG. 11 illustrates another example tubing set including an example cap 710, lens wash tubing 745 and gas supply tubing 740. It can be appreciated that the cap 710 may be designed to securely attach to a water reservoir (not shown) designed to hold varying volumes of water. The cap 710 maybe removably attached to the water reservoir in order to replenish fluid in the water reservoir when it becomes depleted.

Additionally, FIG. 11 illustrates an alternative tubing arrangement whereby a portion of the shared lens wash tube 745 is arranged coaxially within the gas supply tube 740, leaving an annular gap between the tubing portions, such that a gas supply from air pump 215 or an alternate gas source may flow through the portion of tubing 740, around the portion of the lens wash tube 745 and into a gap within the water reservoir, pressurizing the remaining fluid in water reservoir, and forcing the fluid up through the portion of shared lens wash tubing 745 toward the endoscope 100. The flow of gas through the portion of tubing 740, around the portion of the lens wash tube 745 and into a gap within the water reservoir is depicted by the arrows 744, while the flow of fluid up through the portion of shared lens wash tubing 745 toward the endoscope 100 is depicted by the arrow 742.

Further, it can be appreciated that the co-axial arrangement of the lens wash tube 745 and the gas supply tube 740 shown in FIG. 11 may be applied to any combination of the gas supply tubing, lens wash supply tubing, upstream irrigation supply tubing, downstream irrigation supply tubing or any other tubing or components of systems 200, 300.

Additionally, FIG. 11 illustrates that the lens wash tube 745 and the gas supply tube 740 may be arranged both co-axially while also being formed into a helical coil configuration, similar to the helical coil configuration of the lens wash tubing 445 described with respect to FIGS. 4-6. For example, the co-axial, helical coil configuration of the lens wash tube 745 and the gas supply tube 740 shown in FIG. 11 may permit the lens wash tubing 475 and the gas supply tubing 740 to be coiled tightly against itself. When tightly coiled, the lens wash tube 745 and the gas supply tube 740 may still permit water and gas to flow therethrough, respectively, and while also allowing the lens wash tubing 745 and the gas supply tubing 740 to be packaged and stored in a tight, compact configuration. Further, the co-axial, helical coil configuration may remain flexible and elongate when desired, similar to that described as described with respect to FIGS. 4-6. It can be appreciated that the helical co-axial arrangement of the lens wash tube 745 and the gas supply tube 740 shown in FIG. 11 may be applied to any co-axial combination including one or more of the gas supply tubing, lens wash supply tubing, upstream irrigation supply tubing, downstream irrigation supply tubing or any other tubing or components of systems 200, 300.

Moreover, it will be appreciated that a lens wash tube, which has any of a variety of extendable features beyond helical coils, such as corrugated folds or elastic materials, can be co-axially disposed within a gas supply tube, which also has any of a variety of extendable features beyond helical coils, such as corrugated folds or elastic materials. Furthermore, such additional extendable features may be applied to any co-axial combination including one or more of the gas supply tubing, lens wash supply tubing, upstream irrigation supply tubing, downstream irrigation supply tubing or any other tubing or components of systems 200, 300.

It will be apparent to those skilled in the art that various modifications and variations can be made to the disclosed device without departing from the scope of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed disclosure being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A fluid reservoir and tube set arranged and configured to couple to an endoscope for use in an endoscopic procedure, the tube set comprising:
a fluid supply tube wherein the fluid supply tube includes an extendable region positioned along a length of the fluid supply tube, and a lumen in fluid communication with the fluid reservoir; and
wherein the extendable region is configured to shift between a first configuration having a first length and a second extended configuration having a second length longer than the first length.

2. The tube set of claim 1, wherein the extendable region comprises a helical coil in the first configuration and/or wherein the extendable region is biased to the first length.

3. The tube set of claim 2, wherein the helical coil includes a plurality of coils.

4. The tube set of claim 3, wherein each individual coil of the plurality of coils is configured to shift between a coiled configuration in the first configuration and a substantially straight configuration in the second extended configuration.

5. The tube set of claim 1, wherein the fluid supply tube has a circular cross-sectional shape in the extendable region.

6. The tube set of claim 2, wherein the fluid supply tube is configured to return to the first configuration after being extended to the second extended configuration.

7. The tube set of claim 2, wherein the helical coil has an outer circumferential shape, and wherein the outer circumferential shape is substantially cylindrical.

8. The tube set of claim 1, wherein the extendable region comprises a plurality of corrugated folds in the first configuration.

9. The tube set of claim 8, wherein the plurality of corrugated folds includes a first fold positioned adjacent to a second fold, and wherein a portion of the first fold is nested within a portion of the second fold.

10. The tube set of claim 1, wherein the extendable region includes a portion of the fluid supply tube which includes an elastic material.

11. A fluid reservoir and tube set arranged and configured to couple to an endoscope for use in an endoscopic procedure, the fluid reservoir and tube set comprising:
a fluid reservoir configured to contain a fluid;
a gas supply tube, wherein the gas supply tube includes an extendable region positioned along a length of the gas supply tube, and a lumen in fluid communication with the fluid reservoir; and
a fluid supply tube including a lumen in fluid communication with the fluid reservoir; and
wherein the extendable region is configured to shift between a first configuration having a first length and a second extended configuration having a second length longer than the first length.

12. The tube set of claim 11, wherein the extendable region comprises a helical coil in the first configuration and/or wherein the extendable region is biased to the first length.

13. The tube set of claim 12, wherein the helical coil includes a plurality of coils.

14. The tube set of claim 13, wherein each individual coil of the plurality of coils is configured to shift between a coiled configuration in the first configuration and a substantially straight configuration in the second extended configuration.

15. The tube set of claim 11, wherein the extendable region includes a plurality of corrugated folds in the first configuration.

16. The tube set of claim 11, wherein the extendable region includes a portion of the gas supply tube which includes an elastic material.

17. The tube set of claim 11, wherein the fluid supply tube includes an additional extendable region positioned along a length of the fluid supply tube.

18. The tube set of claim 17, wherein at least a portion of the fluid supply tube is co-axial with the gas supply tube and wherein the additional extendable region is co-axial with the extendable region

19. A tube set configured to couple to an endoscope for use in an endoscopic procedure, the tube set comprising:
a fluid reservoir configured to contain a fluid; and
a tubing assembly coupled to the fluid reservoir, the tubing assembly comprising:
a lens wash tube;
a gas supply tube; and
an irrigation tube;
wherein at least one of the lens wash tube, the gas supply tube and the irrigation tube includes an extendable region positioned along a length thereof, and a lumen in fluid communication with the fluid reservoir; and
wherein the extendable region is configured to shift between a first configuration having a first length and a second extended configuration having a second length longer than the first length.

20. The tube set of claim 19, wherein the extendable region comprises a helical coil in the first configuration and/or wherein the extendable region is biased to the first length.
